(19) United States
(12) Patent Application Publication
(10) Pub. No.: US 2004/0023349 A1
(43) Pub. Date: Feb. 5, 2004

(54) Processes for making ethanol

(75) Inventors: Henrik Bisgaard-Frantzen, Bagsvaerd (DK); Lori Henderson, Wake Forest, NC (US)

Correspondence Address: NOVOZYMES NORTH AMERICA, INC., 500 FIFTH AVENUE, SUITE 1600, NEW YORK, NY, 10110, US

(73) Assignees: Novozymes A/S, Bagsvaerd (DK); Novozymes North America, Inc., Franklinton, NC (US)

(21) Appl. No.: 10/460,455

(22) Filed: Jun. 12, 2003

Related U.S. Application Data
(60) Provisional application No. 60/388,488, filed on Jun. 13, 2002.

(51) Int. Cl. C12P 7/06

(52) U.S. Cl. 435/161

(57) ABSTRACT

The present invention provides improved processes for recovering components of distillers' grain, such as, the components of distillers' dried grain (DDG), for use in various applications, including in the production of ethanol.

### CROSS-REFERENCE TO RELATED APPLICATIONS

This application claims the benefit of U.S. provisional application No. 60/388,488 filed Jun. 13, 2002, the contents of which are fully incorporated herein by reference

### FIELD OF THE INVENTION

The present invention relates to processes for making ethanol and other starch-based products.

### BACKGROUND OF THE INVENTION.

The production of ethanol for fuel, beverages and industrial use is a major industry. Ethanol has widespread application, including for use as a gasoline additive or as a straight liquid fuel.

Ethanol manufactures employ two main processes in the production of ethanol and other starch-based products: wet milling and dry milling. Wet milling and dry milling are very different processes and result in very different products and co-products. In wet milling, whole cereal grains are first steeped to loosen the outer fiber and then the grain is separated into the different fractions (starch, germ, fiber, oil and protein). In addition to ethanol, the wet milling process is used to produce a variety of diverse co-products, such as, starch, corn sweeteners, corn oil, high and low protein products and fiber.

In dry milling, whole cereal grains are ground in a substantially dry state, that is, without steeping the kernels to separate the kernels into the major components. The ground meal is then liquefied, saccharified, fermented, and distilled to make ethanol. In addition to ethanol, the other major co-products of dry milling are the spent grains (termed distillers' grains) and carbon dioxide. Distillers' grains are produced from the de-alcoholized fermentation residues which remain after cereal grains have been fermented by yeast. The distillers' grains may be used as an animal feed, but otherwise have a relatively low commercial value compared to the co-products produced in wet milling ethanol production.

Despite the low value of the distillers' grain co-product, dry milling is preferred over wet milling for ethanol production because a dry mill ethanol unit is much less capital and energy intensive than a wet mill ethanol unit. However, because of the relatively low value of the distillers' grain co-product, when raw material prices increase, the net raw material costs associated with dry-milling may become cost prohibitive.

It is therefore desired to improve the efficiency of dry milling ethanol production processes.

### SUMMARY OF THE INVENTION

The present invention provides improved processes for recovering components of distillers' grain for use in various applications. The present invention also provides processes for producing ethanol in a dry milling process by using the components of distillers' grain, such as, the components of distillers' dried grains (DDG), distillers' dried grains with solubles (DDGS) and distillers' wet grains (DWG).

One aspect of the present invention provides processes for recovering components of distillers' grain, preferably, from DDG. In a preferred embodiment of this aspect of the present invention, distillers' grain is treated with a hemi-cellulase and/or a cellulase to release the starch and/or non-starch components (e.g., protein) present in distillers' grain. In a more preferred embodiment of this aspect of the present invention, distillers' grain is also subject to a chemical treatment and/or mechanical treatment to promote the release of starch and/or non-starch components from distillers' grain, preferably prior to or simultaneously with the hemicellulase and/or cellulase treatment of the distillers' grain. The recovered starch and non-starch components may be separated and/or further processed for use, e.g., in ethanol production or as a nutritional supplement.

Another aspect of the present invention provides processes for producing ethanol in which starch present in distillers' grain, preferably, DDG, is recovered from the distillers' grain and used for ethanol production. By utilizing residual starch present in distillers' grain, further improvements in ethanol yield can be obtained, and in particular, starch which was previously unavailable for use in ethanol production can now be utilized. In a preferred embodiment of this aspect of the present invention, distillers' grain, preferably DDG, is treated with a hemicellulase and/or a cellulase to release starch present in distillers' grain. In a more preferred embodiment of this aspect of the present invention, distillers' grain is also subject to a chemical treatment and/or mechanical treatment to promote the release of the starch from distillers' grain, preferably prior to or simultaneously with the hemicellulase and/or cellulase treatment of distillers' grain. The released starch is preferably treated with a starch degrading enzyme, preferably, a raw starch degrading enzyme, so as to convert the starch recovered from distillers' grain to oligosaccharides. The released starch and/or the treated starch are preferably fed into the ethanol process at a suitable location for further ethanol production, such as, at liquefaction, saccharification and/or fermentation steps.

Another aspect of the present invention provides processes for producing ethanol in which non-starch components, preferably protein, present in distillers' grain, more preferably, in DDG, are recovered from distillers' grain and used for ethanol production. Although not limited to any one theory of operation, improvements in ethanol production processes are obtained by recovering protein present in distillers' grain, treating the recovered protein with a protease and using the protease treated protein to improve fermentation efficiency by providing the yeast used for fermentation with improved nutritional benefits so as to thereby reduce fermentation times. In a preferred embodiment of this aspect of the present invention, distillers' grain is treated with a hemicellulase and/or a cellulase to release protein present in distillers' grain. In a more preferred embodiment of this aspect of the present invention, distillers' grain is also subject to a chemical treatment and/or mechanical treatment to promote the release of the protein, preferably prior to or simultaneously with the hemicellulase and/or cellulase treatment. The released protein is preferably treated with a protease, so as to convert the protein to oligopeptides and amino acids. The released protein and/or the protease-treated protein may then be fed into the ethanol process stream at a suitable location, such as, at liquefaction, saccharification and/or fermentation steps, more preferably directly to the fermentation step.

Yet another aspect of the present invention relates to processes for producing ethanol in which both starch and non-starch components (preferably protein) present in distillers' grain are recovered from distillers' grain and used to further ethanol production. In a preferred embodiment of this aspect of the present invention, distillers' grain is treated with a hemicellulase and/or a cellulase to release starch and protein present in distillers' grain. In a more preferred embodiment of this aspect of the present invention, distillers' grain is also subject to a chemical treatment and/or mechanical treatment to promote the release of the starch and protein, preferably prior to or simultaneously with the hemicellulase and/or cellulase treatment. The starch and protein released from distillers' grain are treated with the combination of a starch degrading enzyme, such as, a raw starch degrading enzyme, and a protease. The treated starch and protein may then be fed into the ethanol production stream at a suitable location, preferably during the saccharification and/or fermentation processes.

### BREIF DESCRIPTION OF THE DRAWING

FIG. 1 shows the production of Reducing Sugar (RS) (mg/mL) over time (hours) from acid pretreated DDG (PDDG) after CELLUCLAST™ 1.5L treatment under agitation

FIG. 2 shows the production of Reducing Sugar (RS) (mg/mL) over time (hours) from acid pretreated DDG (PDDG) after CELLUCLAST™ 1.5L/NOVOZYM™ 188 treatment under agitation

### DETAILED DESCRIPTION OF THE INVENTION

Dry milling processes are well-known in the art, and generally involve the process of grinding whole cereal grains in a dry or substantially dry state. The production of ethanol in accordance with a dry milling process generally includes the main process steps of grinding whole cereal grains to produce a meal, and subjecting the meal to liquefaction, saccharification, fermentation and distillation process steps to produce ethanol. Whole corn grains are the preferred raw starting material for ethanol production, however, other cereal grains may also be used, including, for example, milo, wheat and barley.

Liquefaction is the process in which the long chained starch is degraded into oligosaccharides. Liquefaction processes are well-known in the art, and are usually performed by enzymatic or acid hydrolysis. Preferably, liquefaction is preformed by treating the meal with an effective amount of an alpha-amylase, and such processes are well-known in the art. Liquefaction is often carried out at a temperature of about 105 to 110 degrees Celsius for about 5 to 10 minutes followed by a lower temperature holding period of about 1 to 2 hours at 95 degrees Celsius.

Saccharification is the process in which the oligosaccharides resulting from the liquefaction process are converted by hydrolysis to monosaccharide sugars, such as dextrose. The hydrolysis is preferably preformed enzymatically by addition of a glucoamylase, alone or in combination with other enzymes, such as an alpha-glucosidase and/or an acid alpha-amylase. Saccharification processes are also well-known in the art. A full saccharification process may last about 72 hours, and is often carried out at temperatures from about 30 to 65 degrees Celsius. However, it is often more preferred to do a pre-saccharification step, lasting for about 40 to 90 minutes, and then to do a complete saccharification process during fermentation in a process termed simultaneous saccharification and fermentation (SSF) or simultaneous liquefaction, saccharification, and fermentation process (LSF).

In the fermentation step, yeast is added to the mash to ferment sugars to ethanol and carbon dioxide. Preferred yeast includes strains of Saccharomyces spp., more preferably, from Saccharomyces cerevisiae. Commercially available yeast include, e.g., Red Star®/Lesaffre Ethanol Red (available from Red Star/Lesaffre, USA) FALI (available from Fleischmann's Yeast, a division of Burns Philp Food Inc., USA), SUPERSTART (available from Alltech), GERT STRAND (available from Gert Strand AB, Sweden) and FERMIOL (available from DSM Specialties).

Fermentation processes are well-known in the art. Fermentation is generally carried out for about 24 to 96 hours, such as typically 35-60 hours. In preferred embodiments, the temperature is generally between 26-34° C., in particular about 32° C., and the pH is generally from pH 3-6, preferably around pH 4-5. Yeast cells are preferably applied in amounts of 10⁵ to 10¹², preferably from 10⁷ to 10¹⁰, especially 5×10⁷ viable yeast count per ml of fermentation broth. During the ethanol producing phase the yeast cell count should preferably be in the range from 10⁷ to 10¹⁰, especially around 2×10⁸. Further guidance in respect of using yeast for fermentation can be found in, e.g., “The alcohol Textbook” (Editors K. Jacques, T. P. Lyons and D. R. Kelsall, Nottingham University Press, United Kingdom 1999), which is hereby incorporated by reference. In a continuous fermentation process, the fermenting mash will be allowed to flow, or cascade, through several fermentors until the mash is fully fermented and then leaves the final tank. In a batch fermentation process, the mash stays in one fermentor for an effective amount of time, for example, for about 48 hours, before distillation is started.

Distillation is the process of separating ethanol from the fermented mash, preferably, by evaporation. The vapors are preferably driven off by applying direct heat to the fermented mash. The vapors are collected, condensed and recovered as a liquid and may be redistilled to increase the ethanol concentration. Because ethanol has a higher vapor pressure than water, the vaporization of water and ethanol results in a liquid higher in ethanol. Through condensation, a highly concentrate distillate is obtained. Normal distillation results in a liquid with a purity of about 95% vol % ethanol (190 proof). For fuel ethanol, the final proof must approach 200. To accomplish this result, the ethanol is subjected to further dehydration methods.

Stillage is the product which remains after the mash has been converted to sugar, fermented and distilled into ethanol. Stillage can be separated into two fractions, such as, by centrifugation or screening: (1) wet grain (solid phase) and (2) the thin stillage (supernatant). The solid fraction or distillers' wet grain (DWG) can be pressed to remove excess moisture and then dried to produce distillers' dried grains (DDG). After ethanol has been removed from the liquid fraction, the remaining liquid can be evaporated to concentrate the soluble material into condensed distillers' solubles (DS) or dried and ground to create distillers' dried solubles (DDS). DDS is often mixed with DDG to form distillers' dried grain with solubles (DDGS). DDG, DDGS, and DWG are collectively referred to as distillers' grain(s).

As shown in the table below, distillers' grain contains residual amounts of starch and other non-starch components, including protein. The residual starch and non-starch components are generally not accessible for use in ethanol production, as they are bound in the form of distillers' grain.

TABLE
Distillers' Grain Composition

| Components | Average % |
|---|---|
| Carbohydrate | |
| Non-starch glucan | 11.5 |
| Starch | 6.2 |
| Xylan | 10.9 |
| Galactan | 2.3 |
| Arabinan | 7.6 |
| Mannan | 1.5 |
| Total CHO | 40.1 |
| Other Components | |
| Protein | 32.9 |
| Acetyl groups | 1.6 |
| Ash | 2.6 |

In accordance with the present invention, the starch and/or non-starch components of distillers' grain can be used in ethanol production. Preferably, starch and non-starch components of distillers' grain are recovered by enzymatic treatment of distillers' grain and then used in ethanol production, as described herein. More preferably, the starch and/or non-starch components of distillers' grain are recovered by a combination of chemical and/or mechanical treatment and enzymatic treatment processes, and then used in ethanol production, as described herein.

Enzymatic Treatment

In a preferred embodiment of the present invention, distillers' grain, preferably, DDG, is treated with a hemicellulase and/or a cellulase in amounts effective to release residual starch present in distillers' grain. Preferably, starch obtained from distillers' grain is treated with a starch degrading enzyme to convert the starch to oligosaccharides and other forms suitable for ethanol production. Treatment of the starch with the starch degrading enzyme may be carried out simultaneously with or subsequent to the hemicellulase and/or cellulase treatment.

Any hemicellulase suitable for use in releasing the starch and non-starch components from distillers' grain may be used in the present invention. Preferred hemicellulase for use in the present invention include xylanases, arabinofuranosidases, acetyl xylan esterase, glucuronidases, endo-galactanase, mannases, endo or exo arabinases, exo-galactanses, and mixtures thereof. Preferably, the hemicellulase for use in the present invention is an exo-acting hemicellulase, and more preferably, the hemicellulase is an exo-acting hemicellulase which has the ability to hydrolyze hemicellulose under acidic conditions of below pH 7. An example of hemicellulase suitable for use in the present invention includes VISCOZYME™ (available from Novozymes A/S, Denmark). The hemicellulase is added in an amount effective to release starch and non-starch components present in distillers' grain, such as, in amounts from about 0.001 to 0.5% wt. of solids, more preferably, from about 0.05 to 0.5% wt. of solids.

Any cellulase suitable for use in releasing the starch and non-starch components from distillers' grain may be used in the present invention. The cellulase activity used according to the invention may be derived from any suitable origin, preferably, the cellulase is of microbial origin, such as derivable from a strain of a filamentous fungus (e.g., Aspergillus, Trichoderma, Humicola, Fusarium). Preferably, the cellulase composition acts on both cellulosic and lignocellulosic material. Preferred cellulases for use in the present invention include exo-acting celluases and cellobiases, and combinations thereof. More preferably, the treatment involves the combination of an exo-acting cellulase and a cellobiase. Preferably, the cellulases have the ability to hydrolyze cellulose or lignocellulose under acidic conditions of below pH 7. Examples of cellulases suitable for use in the present invention include, for example, CELLULCLAST™ (available from Novozymes A/S), NOVOZYM™ 188 (available from Novozymes A/S) Other commercially available preparations comprising cellulase which may be used include CELLUZYME™, CEREFLO™ and ULTRAFLO™ (Novozymes A/S), LAMINEX™ and SPEZYME™ CP (Genencor Int.) and ROHAMENT™ 7069 W (from Röhm GmbH). The cellulase enzymes are added in amounts effective to release starch and/or non-starch components present in distillers' grain, such as, in amounts from about 0.001 to 0.5% wt. of solids, more preferably, 0.05% to 0.5% wt. of solids.

Any starch degrading enzyme suitable for converting the released starch to a form suitable for ethanol production may be used. Preferably, the starch degrading enzyme is a raw starch hydrolyzing enzyme, that is, an enzyme which is able to hydrolyze alpha 1,4 glucosidic linkages. Preferably the starch degrading enzymes have the ability to hydrolyze starch under acidic conditions of below pH 7. Examples of suitable starch degrading enzymes for use in the present invention include CGTases, alpha-amylases, glucoamylases and combinations thereof.

An example of a CGTases suitable for use in the present invention is TORUZYME™ (available from Novozymes).

Preferred are alpha-amylases of fungal or bacterial origin. More preferably, the alpha-amylase is a Bacillus alpha-amylases, such as, derived from a strain of B. licheniformis, B. amyloliquefaciens, and B. stearothermophilus. Other alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31. Other alpha-amylase variants and hybrids are described in WO 96/23874, WO 97/41213, and WO 99/19467. Other alpha-amylase includes alpha-amylases derived from a strain of Aspergillus, such as, Aspergillus oryzae and Aspergillus niger alpha-amylases. In a preferred embodiment, the alpha-amylase is a acid alpha-amylase. In a more preferred embodiment the acid alpha-amylase is an acid fungal alpha-amylase or an acid bacterial alpha-amylase. More preferably, the acid alpha-amylase is an acid fungal alpha-amylase derived from the genus Aspergillus. In a preferred embodiment, the alpha-amylase is an acid alpha-amylase. The term “acid alpha-amylase” means an alpha-amylase (E.C. 3.2.1.1) which added in an effective amount has activity at a pH in the range of 3.0 to 7.0, preferably from 3.5 to 6.0, or more preferably from 4.0-5.0.

A preferred acid fungal alpha-amylase is a Fungamyl-like alpha-amylase. In the present disclosure, the term “Fungamyl-like alpha-amylase” indicates an alpha-amylase which exhibits a high homology, i.e. more than 50%, 55%, 60%, 65%, 70%, 75%, 80%, 85% or even 90% homology to the amino acid sequence shown in SEQ ID No. 10 in WO96/23874. When used as a maltose generating enzyme fungal alpha-amylases may be added in an amount of 0.001-1.0 AFAU/g DS, preferably from 0.002-0.5 AFAU/g DS, preferably 0.02-0.1 AFAU/g DS.

Preferably the alpha-amylase is an acid alpha-amylase, preferably from the genus Aspergillus, preferably of the species Aspergillus niger. In a preferred embodiment the acid fungal alpha-amylase is the one from A. niger disclosed as “AMYA_ASPNG” in the Swiss-prot/TeEMBL database under the primary accession no. P56271. Also variant of said acid fungal amylase having at least 70% homology, such as at least 80% or even at least 90% identity thereto is contemplated.

Preferred bacterial acid alpha-amylases for use in the present invention may be derived from a strain of the genus Bacillus, preferably B. licheniformis, B. amyloliquefaciens, and B. stearothermophilus. Preferred commercial compositions comprising alpha-amylase include MYCOLASE™ from DSM (Gist Brochades), BAN™, TERMAMYL™ SC, FUNGAMYL™, LIQUOZYME™ X and SAN™ SUPER, SAN™ EXTRA L (Novozymes A/S) and CLARASE™ L-40,000, DEX-LO™, SPEYME FRED, SPEZYME™ AA, and SPEZYME™ DELTA AA (Genencor Int.), and the acid fungal alpha-amylase sold under the trade name SP 288 (available from Novozymes A/S, Denmark).

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic alpha-amylase from B. stearothermophilus strain NCIB 11837 is commercially available from Novozymes A/S under the tradename NOVAMYL™. Maltogenic alpha-amylases are described in U.S. Pat. Nos. 4,598,048, 4,604,355 and 6,162,628, which are hereby incorporated by reference. Preferably, the maltogenic alpha-amylase is used in a raw starch hydrolysis process, as described, e.g., in WO 95/10627, which is hereby incorporated by reference.

The starch degrading enzymes are added in amounts effective to hydrolyze starch obtained from the distillers' grains, such as, in amounts from about 0.001 to 0.5% wt of solids, more preferably, from about 0.05 to 0.5% wt of solids.

The alpha-amylase may be added in amounts as are well-known in the art. When measured in AAU units the acid alpha-amylase activity is preferably present in an amount of 5-50,0000 AAU/kg of DS, in an amount of 500-50,000 AAU/kg of DS, or more preferably in an amount of 100-10,000 AAU/kg of DS, such as 500-1,000 AAU/kg DS. Fungal acid alpha-amylase are preferably added in an amount of 10-10,000 AFAU/kg of DS, in an amount of 500-2,500 AFAU/kg of DS, or more preferably in an amount of 100-1,000 AFAU/kg of DS, such as approximately 500 AFAU/kg DS.

The glucoamylase used according to an embodiment of the process of the invention may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel et al. (1984), EMBO J. 3 (5), p. 1097-1102), or variants thereof, such as disclosed in WO 92/00381 and WO 00/04136; the A. awamori glucoamylase (WO 84/02921), A. oryzae (Agric. Biol. Chem. (1991), 55 (4), p. 941-949), or variants or fragments thereof.

Other Aspergillus glucoamylase variants include variants to enhance the thermal stability: G137A and G139A (Chen et al. (1996), Prot. Engng. 9, 499-505); D257E and D293E/Q (Chen et al. (1995), Prot. Engng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Engng. 10, 1199-1024. Other glucoamylases include Talaromyces glucoamylases, in particular, derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831).

Commercially available compositions comprising glucoamylase include AMG 200L; AMG 300 L; AMG E, SAN™ SUPER, SAN™ EXTRA L, SPIRIZYME™ PLUS, SPIRIZYME™ FUEL, SPIRIZYME™ FG, SPIRIZYME™ E, and (all from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.02-20 AGU/g DS, preferably 0.1-10 AGU/g DS, such as 2 AGU/g DS.

The starch recovered from distillers' grain may be used for additional ethanol production, such as, by feeding the raw starch and/or the treated starch (comprising oligosaccharides) into the main ethanol process stream, such as, for liquefaction, saccharification and/or fermentation. Preferably, treated starch is fed directly into a saccharification, fermentation, SSF or LSF process for further ethanol production. Alternatively, the raw starch or treated starch is fed into a liquefaction process for further ethanol production.

In another preferred embodiment of the present invention, distillers' grain, preferably, DDG, is treated with a hemicellulase and/or a cellulase in amounts effective to recover protein present in distillers' grain. Preferably, the protein recovered from distillers' grain is treated with a protease to convert the protein to oligopeptides and amino acids. The protease treatment may be carried out simultaneously with or subsequent to the hemicellulase and/or cellulase treatment.

Any protease suitable for converting the released protein to forms suitable for ethanol production may be used. The protease treated material provides nutrition to the yeast. Preferred proteases for use in the present invention have the ability to hydrolyze proteins under acidic conditions below pH 7.

Suitable proteases include fungal and bacterial proteases. Preferred proteases are acidic proteases, i.e., proteases characterized by the ability to hydrolyze proteins under acidic conditions below pH 7. Suitable acid fungal proteases include fungal proteases derived from Aspergillus, Mucor, Rhizopus, Candida, Coriolus, Endothia, Enthomophtra, Irpex, Penicillium, Sclerotium and Torulopsis. Especially contemplated are proteases derived from Aspergillus niger (see, e.g., Koaze et al., (1964), Agr. Biol. Chem. Japan, 28, 216), Aspergillus saitoi (see, e.g., Yoshida, (1954) J. Agr. Chem. Soc. Japan, 28, 66), Aspergillus awamori (Hayashida et al., (1977) Agric. Biol. Chem., 42(5), 927-933, Aspergillus aculeatus (WO 95/02044), or Aspergillus oryzae; and acidic proteases from Mucor pusillus or Mucor miehei.

Bacterial proteases, which are not acidic proteases, include the commercially available products ALCALASE™ and NEUTRASE™ (available from Novozymes A/S). Other proteases include GC106 and SPEZYME FAN (available from Genencor Int, Inc., USA and NOVOZYM™ 50006, NOVOREN™ and FLAVORZYM™ (all available from Novozymes A/S, Denmark).

Preferably, the protease is an aspartic acid protease, as described, for example, Handbook of Proteolytic Enzymes, Edited by A. J. Barrett, N. D. Rawlings and J. F. Woessner, Academic Press, San Diego, 1998, Chapter 270). Suitable examples of aspartic acid protease include, e.g., those disclosed in R. M. Berka et al. Gene, 96, 313 (1990)); (R. M. Berka et al. Gene, 125, 195-198 (1993)); and Gomi et al. Biosci. Biotech. Biochem. 57, 1095-1100 (1993), which are hereby incorporated by reference.

The proteases are added in amounts effective to convert protein obtained from the distillers' grain to oligopeptides and amino acids, such as, in amounts from about 0.001 to 0.5% wt. of solids, more preferably, about 0.05 to 0.5% wt of solids.

The recovered protein is preferably used in ethanol production, such as, by feeding the protease treated protein (comprising oligopeptides and amino acids) into the main ethanol process stream to improve fermentation efficiency by providing the yeast with improved nutritional benefits. Preferably, the protease treated protein is fed directly into the saccharification, fermentation, SSF or LSF process to further ethanol production. Alternatively, the released protein or protease treated protein are fed into the liquefaction process.

In another preferred embodiment, the process entails a method for recovering both starch and protein from distillers' grain, preferably, from DDG, and using the starch and protein for further ethanol production. In accordance with this embodiment of the present invention, distillers' grain, preferably, DDG, is treated with a hemicellulase and/or a cellulase in amounts effective to release starch and protein present in distillers' grain. The starch obtained from the distillers' grain is treated with a starch degrading enzyme, more preferably, a raw starch degrading enzyme, and the protein obtained from distillers' grain is treated with a protease. Treatment of the released starch and protein may be carried out simultaneously with or as separate process steps, as desired. The process preferably further entails feeding the treated starch and protein to the ethanol production stream, such as, to the liquefaction, saccharification or fermentation steps, more preferably to the saccharification and/or fermentation steps, and most preferably to an SSF or LSF process.

Chemical and/or Mechanical Treatment

In preferred embodiments of the present invention, chemical treatment and/or mechanical treatment processes are used in combination with the enzymatic processes described herein to promote the release of starch and non-starch components from distillers' grain, that is, to further the release of the starch and non-starch components from distillers' grain or to further the enzymatic processes described herein. Preferably, the chemical and/or mechanical treatment processes are carried out prior to the enzymatic processes in a pre-treatment process so as to improve the enzymatic processes described herein. In the alternative, the chemical and/or mechanical treatment processes are carried out simultaneously with the enzymatic processes, such as simultaneously with the hemicellulase and/or cellulase treatment described herein.

As used in the present invention, “a chemical treatment process” refers to any chemical treatment process which can be used to promote the release of starch and/or non-starch components (in particular, protein) from distillers' grains. Examples of chemical treatments suitable for use in the present invention include, for example, acid treatment, wet oxidation, and solvent treatment. More preferably, the chemical treatment process is an acid treatment process, more preferably, a continuous dilute or mild acid treatment, such as, treatment with sulfuric acid, or another organic acid, such as acetic acid, citric acid, tartaric acid, succinic acid, or mixtures thereof. Other acids may also be used. Mild acid treatment means in the context of the invention that the treatment pH lies in the range from 1 to 5, preferably 1 to 3. In a specific embodiment the acid concentration is in the range from 0.5 to 1.7 wt. % sulfuric acid. Wet oxidation techniques involve the use of oxidizing agents such as, sulfite based oxidizing agents and the like. Examples of solvent treatments include treatment with DMSO (Dimethyl Sulfoxide) and the like. Chemical treatment processes are generally carried out for about 5 to about 10 minutes.

As used in the present invention, the phrase “a mechanical treatment process” refers to any mechanical treatment process which can be used to promote the release of starch and non-starch components (in particular, protein) from distillers' grains, in particular, from DDG. Preferably, a mechanical treatment process involves a process which uses high pressure and high temperature to promote the release of the starch and non-starch components in distillers' grain. In context the in invention high pressure means pressure in the range from 300 to 600, preferably 400 to 500, such as around 450 psi. In context the in invention high temperature means pressure in the range from 100 to 300° C., preferably from 140 to 235° C. In a specific embodiment the impregnation is carried out at a pressure of about 450 psi and at a temperature of 235° C. More preferably, the mechanical process is a batch-process, steam gun hydrolyzer system which uses high pressure and high temperature, such as, using the Sunds Hydrolyzer (available from Sunds Defibrator AB (Sweden).

In preferred embodiments, both chemical and mechanical treatment is carried out, for example, involving, for example, both mild acid treatment and high temperature and pressure treatment. The chemical and mechanical processes may be carried out sequentially or simultaneously, as desired.

Accordingly, in a more preferred embodiment, the process comprises the steps of (a) pre-treating distillers' grain, preferably, DDG, with a chemical treatment and/or a mechanical treatment to promote the release of starch and protein present in distillers' grain, (b) treating the chemically and/or mechanically treated distillers' grain with a hemicellulase and/or cellulase to further promote the release of starch and protein from distillers' grain and (c) treating the released starch and protein with a starch degrading enzyme and a protease. Preferably, the process further comprises the step of feeding the treated starch and protein into the ethanol process stream, more preferably at the liquefaction, saccharification and/or fermentation steps, and most preferably at the saccharification and/or fermentation steps, such as, an SSF process or LSF process.

The enzymatic treatment is carried out in a suitable aqueous environment, which can be readily determined by one skilled in the art practicing the present invention. Any suitable process time, holding time, temperature and pH conditions may be employed, which can be readily determined by one skilled in the art practicing the present invention. Preferably, the enzymatic treatment processes is from about 2 to about 60 hours. The temperature of the enzymatic treatment processes are preferably from about 40 to 60 degrees Celsius. The pH of aqueous treatment solution used in the enzymatic treatment process is about preferably from about 4 to about 7, more preferably 4 to 5.

Various modifications of the invention described herein will become apparent to those skilled in the art. Such modifications are intended to fall within the scope of the appending claims.

### Materials and Methods

Materials:

Glucoamylase: SPIRIZYME™ PLUS, available from Novozymes A/S, Denmark

Fungal alpha-amylase: SP288 available from Novozymes A/S, Denmark

Cellulase: CELLUCLAST™ 1.5L available from Novozymes A/S, Denmark

Cellobiase: NOVOZYM™ 188 (available from Novozymes A/S, Denmark)

Protease: NOVOZYM 50006 (available from Novozymes A/S, Denmark)

Yeast: Ethanol Red available from Red Star/Lesaffre, USA

Methods:

RS Determination: Reducing Sugar Assay: PHBAH

1. Prepare glucose standards from a stock 2 mg/mL (10mM) solution. Dilute in Carbonate/Bicarbonate buffer to give standards of 0.1, 0.075, 0.05, 0.0375, 0.025, 0.0125 and 0.0075 mg/mL. Standards can be aliquotted and frozen at −40° C. for up to 3 weeks.

2. Dilute samples 1:10 in Carbonate/Bicarbonate by adding 30 μL of sample to 270 microL of buffer. Mix. Additional dilutions are made serially from the initial 1:10 dilution into Carbonate/Bicarbonate as necessary to get the test response to fall on the standard curve.

3. Into the wells of a 0.2 mL PCR plate (Greiner), transfer 100 μL of each sample, standard and blank. Standards are analyzed in duplicate, samples in triplicate.

4. Add 50 microL of PHBAH reagent to each well. Seal plate with a Mylar sealer.

5. Place the PCR plate in a 90° C. heat block for 10 minutes.

6. Retrieve plate and allow cooling to ambient temperature.

7. Transfer 50 microL of each sample, standard and blank to a corresponding well of a 96 well assay plate (Nunc) containing 50 microL of PHBAH buffer (1:2 dilution).

8. Read absorbance at 405 nm.

Determination of FAU Activity

One Fungal Alpha-Amylase Unit (FAU) is defined as the amount of enzyme, which breaks down 5.26 g starch (Merck Amylum solubile Erg. B.6, Batch 9947275) per hour based upon the following standard conditions:

| | |
|---|---|
| Substrate | Soluble starch |
| Temperature | 37° C. |
| PH | 47 |
| Reaction time | 7-20 minutes |

Determination of Acid Alpha-Amylase Activity (AFAU)

Acid alpha-amylase activity is measured in AFAU (Acid Fungal Alpha-amylase Units), which are determined relative to an enzyme standard.

The standard used is AMG 300 L (from Novozymes A/S, Denmark, glucoamylase wildtype Aspergillus niger G1, also disclosed in Boel et al. (1984), EMBO J. 3 (5), p. 1097-1102) and WO 92/00381). The neutral alpha-amylase activity in this AMG falls after storage at room temperature for 3 weeks from approx. 1 FAU/mL to below 0.05 FAU/mL.

The acid alpha-amylase activity in this AMG standard is determined in accordance with the following description. In this method, 1 AFAU is defined as the amount of enzyme, which degrades 5.260 mg starch dry matter per hour under standard conditions.

Iodine forms a blue complex with starch but not with its degradation products. The intensity of colour is therefore directly proportional to the concentration of starch. Amylase activity is determined using reverse colorimetry as a reduction in the concentration of starch under specified analytic conditions.

| | | |
|---|---|---|
| | Alpha-amylase | |
| Starch + Iodine | → | Dextrins + Oligosaccharides |
| | 40° C., pH 2.5 | |
| Blue/violet | t = 23 sec. | Decoloration |

Standard conditions/reaction conditions: (per minute)

| | |
|---|---|
| Substrate: | Starch, approx. 0.17 g/L |
| Buffer: | Citrate, approx. 0.03 M |
| Iodine (I₂): | 0.03 g/L |
| CaCl_{2:} | 1.85 mM |
| pH: | 2.50 ± 0.05 |
| Incubation temperature: | 40° C. |
| Reaction time: | 23 seconds |
| Wavelength: | lambda = 590 nm |
| Enzyme concentration: | 0.025 AFAU/mL |
| Enzyme working range: | 0.01-0.04 AFAU/mL |

If further details are preferred these can be found in EB-SM-0259.02/01 available on request from Novozymes A/S, Denmark, and incorporated by reference.

Acid Alpha-Amylase Units (AAU)

The acid alpha-amylase activity can be measured in AAU (Acid Alpha-amylase Units), which is an absolute method. One Acid Amylase Unit (AAU) is the quantity of enzyme converting 1 g of starch (100% of dry matter) per hour under standardized conditions into a product having a transmission at 620 nm after reaction with an iodine solution of known strength equal to the one of a color reference.

Standard conditions/reaction conditions:

| | |
|---|---|
| Substrate: | Soluble starch. Concentration approx. |
| | 20 g DS/L. |
| Buffer: | Citrate, approx. 0.13 M, pH = 4.2 |
| Iodine solution: | 40.176 g potassium iodide + 0.088 g iodine/L |
| City water | 15°-20° dH (German degree hardness) |
| pH: | 4.2 |
| Incubation temperature: | 30° C. |
| Reaction time: | 11 minutes |
| Wavelength: | 620 nm |
| Enzyme concentration: | 0.13-0.19 AAU/mL |
| Enzyme working range: | 0.13-0.19 AAU/mL |

The starch should be Lintner starch, which is a thin-boiling starch used in the laboratory as calorimetric indicator. Lintner starch is obtained by dilute hydrochloric acid treatment of native starch so that it retains the ability to color blue with iodine. Further details can be found in EP0140410B2, which disclosure is hereby included by reference.

Glucoamylase Activity (AGI)

Glucoamylase (equivalent to amyloglucosidase) converts starch into glucose. The amount of glucose is determined here by the glucose oxidase method for the activity determination. The method is described in the section 76-11 Starch-Glucoamylase Method with Subsequent Measurement of Glucose with Glucose Oxidase in “Approved methods of the American Association of Cereal Chemists”. Vol.1-2 AACC, from American Association of Cereal Chemists, (2000); ISBN: 1-891127-12-8.

One glucoamylase unit (AGI) is the quantity of enzyme which will form 1 micromol of glucose per minute under the standard conditions of the method.

Standard conditions/reaction conditions:

| | |
|---|---|
| Substrate: | Soluble starch. |
| | Concentration approx. 16 g dry matter/L. |
| Buffer: | Acetate, approx. 0.04 M, pH = 4.3 |
| pH: | 4.3 |
| Incubation temperature: | 60° C. |
| Reaction time: | 15 minutes |
| Termination of the reaction: | NaOH to a concentration of approximately |
| | 0.2 g/L (pH˜9) |
| Enzyme concentration: | 0.15-0.55 AAU/mL. |

The starch should be Lintner starch, which is a thin-boiling starch used in the laboratory as colorimetric indicator. Lintner starch is obtained by dilute hydrochloric acid treatment of native starch so that it retains the ability to color blue with iodine.

Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate concentration of 23.2 mM, acetate buffer 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| | |
|---|---|
| AMG incubation: | |
| Substrate: | maltose 23.2 mM |
| Buffer: | acetate 0.1 M |
| pH: | 4.30 ± 0.05 |
| Incubation | 37° C. ± 1 |
| temperature: | |
| Reaction time: | 5 minutes |
| Enzyme working range: | 0.5-4.0 AGU/mL |
| Color reaction: | |
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation | 37° C. ± 1 |
| temperature: | |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

Determination of Cellobiase Activity (CBU)

Cellobiase (beta-glucosidase EC 3.2.1.21) hydrolyzes beta-1,4 bonds in cellobiose to release two glucose molecules. The amount of glucose released is determined specifically and quantitatively using the hexokinase method as follows:

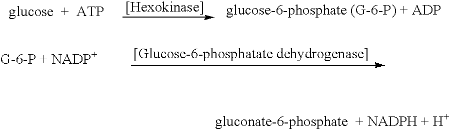

The increase in absorbance is then measured at 340 nm as the absorbance value for NADPH is high at this wavelength.

Reaction conditions

| | |
|---|---|
| Reaction: | |
| Temperature: | 40° C. |
| pH: | 5.0 |
| Detection: | |
| Reaction time: | 15 minutes |
| Wavelength: | 340 nm |

One cellobiase unit (CBU) is the amount of enzyme, which releases 2 micro mole glucose per minute under the standard conditions above with cellobiose as substrate.

A folder (EB-SM-0175.02/02) describing this analytical method in more detail is available on request from Novozymes A/S, Denmark, which folder is hereby included by reference.

Measurement of Cellulase Activity Using Filter Paper Assay (FPU Assay)

1. Source of Method

1.1 The method is disclosed in a document entitled “Measurement of Cellulase Activities” by Adney, B. and Baker, J. 1996. Laboratory Analytical Procedure, LAP-006, National Renewable Energy Laboratory (NREL). It is based on the IUPAC method for measuring cellulase activity (Ghose, T. K., Measurement of Cellulse Activities, Pure & Appl. Chem. 59, pp. 257-268, 1987.

2. Procedure

2.1 The method is carried out as described by Adney and Baker, 1996, supra, except for the use of a 96 well plates to read the absorbance values after color development, as described below.

2.2 Enzyme Assay Tubes:

2.2.1 A rolled filter paper strip (#1 Whatman; 1×6 cm; 50 mg) is added to the bottom of a test tube (13×100 mm).

2.2.2 To the tube is added 1.0 mL of 0.05 M Na-citrate buffer (pH 4.80).

2.2.3 The tubes containing filter paper and buffer are incubated 5 min. at 50° C. (±0.1° C.) in a circulating water bath.

2.2.4 Following incubation, 0.5 mL of enzyme dilution in citrate buffer is added to the tube. Enzyme dilutions are designed to produce values slightly above and below the target value of 2.0 mg glucose.

2.2.5 The tube contents are mixed by gently vortexing for 3 seconds.

2.2.6 After vortexing, the tubes are incubated for 60 mins. at 50° C. (±0.1° C.) in a circulating water bath.

2.2.7 Immediately following the 60 min. incubation, the tubes are removed from the water bath, and 3.0 mL of DNS reagent is added to each tube to stop the reaction. The tubes are vortexed 3 seconds to mix.

2.3 Blank and Controls

2.3.1 A reagent blank is prepared by adding 1.5 mL of citrate buffer to a test tube.

2.3.2 A substrate control is prepared by placing a rolled filter paper strip into the bottom of a test tube, and adding 1.5 mL of citrate buffer.

2.3.3 Enzyme controls are prepared for each enzyme dilution by mixing 1.0 mL of citrate buffer with 0.5 mL of the appropriate enzyme dilution.

2.3.4 The reagent blank, substrate control, and enzyme controls are assayed in the same manner as the enzyme assay tubes, and done along with them.

2.4 Glucose Standards

2.4.1 A 100 mL stock solution of glucose (10.0 mg/mL) is prepared, and 5 mL aliquots are frozen. Prior to use, aliquots are thawed and vortexed to mix.

2.4.2 Dilutions of the stock solution are made in citrate buffer as follows:

G1=1.0 mL stock+0.5 mL buffer=6.7 mg/mL=3.3 mg/0.5 mL

G2=0.75 mL stock+0.75 mL buffer=5.0 mg/mL=2.5 mg/0.5 mL

G3=0.5 mL stock+1.0 mL buffer=3.3 mg/mL=1.7 mg/0.5 mL

G4=0.2 mL stock+0.8 mL buffer=2.0 mg/mL=1.0 mg/0.5 mL

2.4.3 Glucose standard tubes are prepared by adding 0.5 mL of each dilution to 1.0 mL of citrate buffer.

2.4.4 The glucose standard tubes are assayed in the same manner as the enzyme assay tubes, and done along with them.

2.5 Color Development

2.5.1 Following the 60 min. incubation and addition of DNS, the tubes are all boiled together for 5 mins. in a water bath.

2.5.2 After boiling, they are immediately cooled in an ice/water bath.

2.5.3 When cool, the tubes are briefly vortexed, and the pulp is allowed to settle. Then each tube is diluted by adding 50 microL from the tube to 200 microL of ddH20 in a 96-well plate. Each well is mixed, and the absorbance is read at 540 nm.

2.6 Calculations (examples are given in the NREL document)

2.6.1 A glucose standard curve is prepared by graphing glucose concentration (mg/0.5 mL) for the four standards (G1-G4) vs. A₅₄₀. This is fitted using a linear regression (Prism Software), and the equation for the line is used to determine the glucose produced for each of the enzyme assay tubes.

2.6.2 A plot of glucose produced (mg/0.5 mL) vs. total enzyme dilution is prepared, with the Y-axis (enzyme dilution) being on a log scale.

2.6.3 A line is drawn between the enzyme dilution that produced just above 2.0 mg glucose and the dilution that produced just below that. From this line, it is determined the enzyme dilution that would have produced exactly 2.0 mg of glucose.

2.6.4 The Filter Paper Units/mL (FPU/mL) are calculated as follows:

FPU/mL=0.37/enzyme dilution producing 2.0 mg glucose

### EXAMPLES

### Example 1

Acid Pretreatment of DDG

Prepare a 10% DS solution of DDG and pretreat in a 1L Parr reactor. The solution is made by weighing out 62.4 g dry weight of DDG, 7.5 g sulfuric acid (72 wt %) and 562 g of water. The final pH is between 1.3 and 1.5. The solids are then heated to 150° C. for 10 minutes. The resulting slurry is washed in a buchner funnel filtration system until the solution in the vacuum flask reaches pH of 4.5. The acid pretreated DDG is referred to as “PDDG”.

Enzymatic treatment: A 20 mg/ml solution of the washed pretreated PDDG was then treated with between:

0.001-0.01 FPU CELLUCLAST™ 1.5L /mg PDDG

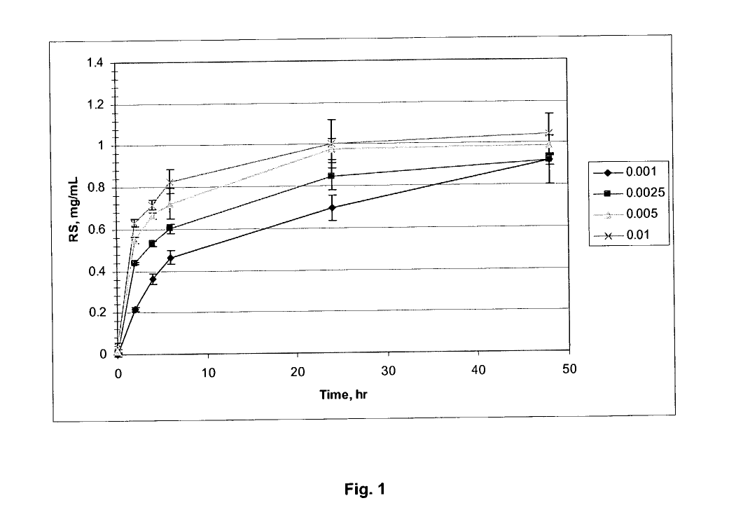

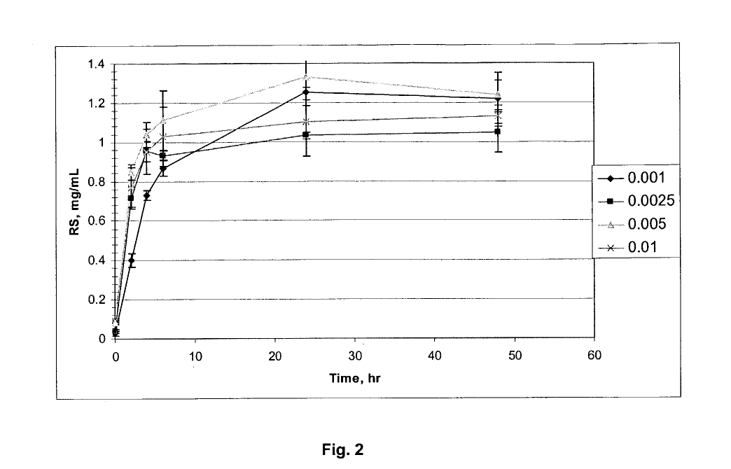

0.001-0.01 FPU CELLUCLAST™ 1.5/mg PDDG L+0.064 CBU/mg PDDG of NOVOZYM™ 188 at a pH of 5.0 up to 72 hours at 32-50° C. A 20 microL sample is removed for RS determination. Reducing Sugar (RS) was determined using the method descried in the “Materials and Methods” section. The result of the test is shown in FIG. 1 and 2

### Example 2

Production of Ethanol from Pretreated DDG

A 15% DS cellulase pretreated PDDG slurry is prepared as described in Example 1 and introduced into shake flasks for LSF fermentation. The shake flasks have a 2:5 medium to flask volume ratio and are equipped with water trap. The pH of the 15% DS PDDG slurry is adjusted to pH 5 with phosphoric acid. Ethanol Red yeast is propagated aerobically at 500 rpm and 32° C. for 8 hours with 0.02% DS NOVOZYM™ 50006 (protease). SP 288 (fungal acid alpha-amylase), 0.8 AFAU/g DS, SPIRIZYME™ FUEL (glucoamylase), 2 AGU/g DS and yeast propagate are introduced into the slurry immediately before filling the shake flasks. The fermentation is carried out at 32° C. for 64 hours. The ethanol percentage is determined by HPLC and compared to DDG fermentation (Control).

### Example 3

Production of Ethanol from Pretreated DDG Via an SSF Process

A mixture of 240 grams of corn mash and 10 grams PDDG is prepared and introduced into 500 ml shake flasks for fermentation equipped with a trap. The pH of the slurry is adjusted to pH 5 with phosphoric acid. Ethanol Red yeast is propagated aerobically at 500 rpm and 32° C. for 8 hours and 0.02% DS NOVOZYM™ 50006 (Protease). SP 288 (fungal acid alpha-amylase), 0.8 AFAU/g DS, SPIRIZYME™ FUEL (Glucoamylase), 2 AGU/g DS and yeast propagate are introduced into the slurry immediately before filling the shake flasks. The fermentation is carried out at 32° C. for 64 hours. The ethanol percentage is determined by HPLC and compared to a corresponding fermentation comprising 240 grams of corn mash in a 500 ml shake flask (control).

1. A process for producing ethanol, comprising:
(a) treating distillers' grain with a hemicellulase and/or cellulase to release starch present in the distillers' grain; and
(b) using the released starch to produce ethanol.

2. The process of claim 1, wherein the distillers' grain is further subject to a chemical treatment and/or a mechanical treatment.

3. The process of claim 1, wherein the distillers' grain is further subject to a chemical treatment and/or a mechanical treatment prior to step (a).

4. The process of claims 2 or 3, wherein the chemical treatment comprises treating the distillers' grain with a mild acid.

5. The process of claims 2 or 3, wherein the mechanical treatment comprises treating the distillers' grain with a high temperature and a high pressure.

6. The process of any of the proceeding claims, wherein step (b) comprises treating the starch with a starch degrading enzyme and feeding the treated starch into a liquefaction, saccharfication or fermentation process.

7. The process of any of the proceeding claims, wherein step (b) comprises treating the starch with a raw starch degrading enzyme, and feeding the treated starch into a liquefaction, saccharfication or fermentation process.

8. The process of any of the proceeding claims, wherein step (b) comprises treating the starch with an alpha-amylase and feeding the treated starch into a liquefaction, saccharfication or fermentation process.

9. The process of any of the proceeding claims, wherein step (b) comprises treating the starch with a CGTase and feeding the treated starch into a liquefaction, saccharfication or fermentation process.

10. The process of any of the proceeding claims, wherein step (b) comprises treating the starch with a glucoamylase and feeding the treated starch into a liquefaction, saccharfication or fermentation process.

11. The process of any of the proceeding claims, wherein step (b) comprises treating the starch with an enzyme selected from the group consisting of an alpha-amylase, a CGTase, a glucoamylase, and combinations thereof and feeding the treated starch into a liquefaction, saccharfication or fermentation process.

12. The process of claim 1, wherein step (b) comprises feeding the starch into a liquefaction process.

13. The process of any of the proceeding claims, wherein the distillers' grain is distillers' dried grain.

14. The process of any of the proceeding claims, wherein the distillers' grain is distillers' dried grain with solubles.

15. The process of any of the proceeding claims, wherein the distillers' grain is distillers' wet grain.

16. A process for producing ethanol, comprising:
(a) treating distillers' grain with a hemicellulase and/or cellulase to release protein present in the distillers' grain; and
(b) using the released protein in the ethanol production process.

17. The process of claim 16, wherein the distillers' grain is further subject to a chemical treatment and/or a mechanical treatment.

18. The process of claim 16, wherein the distillers' grain is further subject to a chemical treatment and/or a mechanical treatment prior to step (a).

19. The process of claims 17 or 18, wherein the chemical treatment comprises treating the distillers' grain with a mild acid.

20. The process of claim 17 or 18, wherein the mechanical treatment comprises treating the distillers' grain with a high temperature and a high pressure.

21. The process of any of the proceeding claims, wherein step (b) comprises treating the protein with a protease and feeding the protease treated protein into a liquefaction, saccharification or fermentation process.

22. The process of claim 16, wherein step (b) comprises feeding the protein into a liquefaction process.

23. The process of claim 21, wherein the protease treated protein is fed into a simultaneous saccharification and fermentation process.

24. The process of any of the proceeding claims, wherein the distillers' grain is distillers' dried grain.

25. The process of any of the proceeding claims, wherein the distillers' grain is distillers' dried grain with solubles.

26. The process of any the proceeding claims, wherein the distillers' grain is distillers' wet grain.

27. A process for producing ethanol, comprising:
(a) treating distillers' grain with a hemicellulase and/or cellulase to release starch and protein present in the distillers' grain; and
(b) using the released starch and protein to produce ethanol.

28. The process of claim 27, wherein the distillers' grain is further subject to a chemical treatment and/or a mechanical treatment.

29. The process of claim 27, wherein the distillers' grain is further subject to a chemical treatment and/or a mechanical treatment prior to step (a).

30. The process of claims 28 or 29, wherein the chemical treatment comprises treating the distillers' grain with a mild acid.

31. The process of claim 28 or 29, wherein the mechanical treatment comprises treating the distillers' grain with a high temperature and a high pressure.

32. The process of any of the proceeding claims, wherein step (b) comprises treating the starch with an alpha-amylase and treating the protein with a protease, and feeding the treated starch and treated protein into a liquefaction, saccharfication or fermentation process.

33. The process of claim 32, wherein the treated starch and the treated protein are fed into a simultaneous saccharification and fermentation process (SSF) or into a simultaneous liquefaction, saccharification, and fermentation process (LSF)

34. The process of any of the proceeding claims, wherein the distillers' grain is distillers' dried grain.

35. The process of any of the proceeding claims, wherein the distillers' grain is distillers' dried grain with solubles.

36. The process of any of the proceeding claims, wherein the distillers' grain is distillers' wet grain.

37. A process for producing ethanol, comprising:
(a) pre-treating distillers' grain with a chemical treatment and/or a mechanical treatment to release starch and protein present in distillers' grain;
(b) treating the chemically and/or mechanically treated distillers' grain with a hemicellulase and/or cellulase to further release of starch and protein from distillers' grain;
(c) treating the released starch and protein with a starch degrading enzyme and a protease; and
(d) feeding the treated starch and protein into and ethanol production process.

38. Use of distillers' grain in making ethanol.

39. Use of distillers' dried grain in making ethanol.

40. Use of distillers' dried grain with solubles in making ethanol.

41. Use of distillers' wet grain in making ethanol.

42. Use of starch present in distillers' grain in making ethanol.

43. Use of protein present in distillers' grain in making ethanol.
